(19) United States
(12) Patent Application Publication
(10) Pub. No.: US 2019/0060589 A1
(43) Pub. Date: Feb. 28, 2019

(54) Lighted Tubing and Walker

(71) Applicant: Alexis Baird, Hanover, MA (US)

(72) Inventor: Alexis Baird, Hanover, MA (US)

(21) Appl. No.: 15/693,461

(22) Filed: Aug. 31, 2017

(51) Int. Cl. A61M 16/00; A61M 16/06; A61M 16/08; A61B 90/30

(52) U.S. Cl. CPC A61M 16/0003; A61M 16/0672; A61M 16/0875; A61B 90/30; A61M 2205/8293; A61M 2205/3368; A61M 2205/3553; A61M 2205/587; A61M 2205/8206; A61M 2205/3306

(57) ABSTRACT

The present invention is a device that illuminates oxygen tubing and a walker to make it safer in dark conditions. The device includes a motion sensing device for automatically turning on the lights when there is movement of the walker, and a timer for turning off the lights after the walker has remained stationary for a predetermined amount of time. The device automatically turns the lights on when surrounding conditions are dim through the use of a photo sensor. In addition, the device includes a power switch for manually turning the lights on or off.

Furthermore, the device contains a GPS transmitter and a wireless communication device. This allows a patient to obtain help by manually activating a panic switch. A built in temperature sensor will automatically call for help, if the ambient air temperature becomes too hot due to adverse weather, fire, or other conditions.

### CROSS-REFERENCE TO RELATED APPLICATION

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR

Not Applicable

### BACKGROUND

Unless otherwise indicated herein, the materials described in this section are not prior art to the claims in this application and are not admitted to be prior art by inclusion in this section.

### 1. Field of the Invention

The present invention generally relates to medical devices. More specifically, the present invention relates to illuminated medical devices, which includes illuminated oxygen tubing and attachments for a walker or wheelchair.

### 2. Description of Related Art

Having an ailment that makes ambulating difficult or having a loved one that you worry about falling is extremely difficult. In addition to the emotional toll it has on families and patients, treating fall injuries is also very costly. Because the U.S. population is aging, both the number of falls and the costs to treat fall injuries are likely to rise. Every second of every day in the United States an older adult falls, making falls the number one cause of injuries and deaths from injury among older Americans.

The National Council On Aging (“NCOA”) estimates that 1 in 4 Americans aged 65 and over falls every year. Falls result in more than 2.8 million injuries treated in emergency departments annually, including over 800,000 hospitalizations and more than 27,000 deaths. In 2013, the total cost of injuries due to falls was $34 billion. The financial toll for older adult falls is expected to increase as the population ages and may reach $67.7 billion by 2020. Falls, with or without injury, also carry a heavy quality of life impact. A growing number of older adults fear falling and, as a result, limit their activities and social engagements. This can result in further physical decline, depression, social isolation, and feelings of helplessness.

Osteoporosis is a disease that weakens bones and makes elders especially more at risk for broken bones and fractures. The chances of breaking one's hip increases as the person gets older. The average hospital cost for a fall injury is over $30,000 and is going up as the baby boomer generation ages. Often times, a fall will predicate a downward spiral for an elderly person, setting off a cascade of events that they will never bounce back from. Most falls are caused by a combination of risk factors. The more risk factors a person has, the greater their chances of falling. In addition to the elderly, other persons are susceptible to falls, including those that have problems with vision, ambulating, and balance, which is sometimes due to taking medication that makes them feel unstable while walking. Still others are susceptible to falls due to the lack of devices within their home that assist handicapped people with standing, sitting, walking, and avoiding obstacles. The NCOA emphasizes the value of home safety. Adding things like grab bars, raised toilet seats, and shower tubs are all great ideas.

Patients stay overnight at hospitals when in serious condition. These patients may have the freedom to move around while in their hospital room and in the hallways of the hospital. Some of these patients may require oxygen tubing for a sufficient supply of oxygen if the patient is having difficulty breathing. This oxygen tubing connects between a patients nostrils and an oxygen tank or wall source. This oxygen tubing may create a trip hazard. Other patients may require the use of a wheelchair or a walker to aid them in moving around while in their hospital room or in the hallways of the hospital. However, the lights of the hospital hallway or room may be dimmed or off at during night hours. This may lead to accidents if a patient were to move around without being able to navigate around obstacles. For example, patients may trip over their own oxygen cord. Although patients may remove their oxygen tubing while using the restroom, they may forget to put it back on after returning to bed. The faculty of the hospital may be unaware that a patient forgot to reattach their oxygen supply due to the lights being dimmed at night or turned off.

Accordingly, there is a need for a device to allow patients to move safely with oxygen tubing when the lights are dimmed or turned off. There is also a need for hospital faculty to keep better track of their patients during nighttime hours. The device described in this patent application fulfill at least one of these needs or creates other utility.

### BRIEF SUMMARY OF THE INVENTION

It is a principal object to solve at least one of the disadvantages with other attempted solutions or to create other utility by providing a device to allow patients to move safely when the lights are dimmed or off, or to allow hospital faculty to keep better track of their patients during night hours, or both.

One objective of the present invention is to provide illuminated medical devices which aid the faculty of hospitals to keep better track of their patients during night hours. The present invention comprises an illuminated oxygen tubing to aid the hospital faculty to check on their patients. If a patient had accidentally removed the oxygen tubing during night hours, the faculty of the hospital would be aware of this. The present invention also includes illuminated attachments for a walker or wheelchair.

The illuminated attachments may be placed on the oxygen tubing or anywhere on the walker, wheelchair, or a similar device. The illuminated attachments make it less likely that a patient will trip over the oxygen tubing or another obstruction or hazard. The illuminated attachments also make the hospital faculty more aware of the movement of their patients, who may choose to move around during nighttime hours. This increased illumination may reduce the amount of accidents which occur at night in a hospital.

The invention described herein is part of a smarter home for those who are assisted with oxygen. The invention comprises detachable, yet sturdy, lights that attach to any form of assistive device, such as the oxygen tubing that a person may have connected between their nostrils and an oxygen tank or wall source. By illuminating the oxygen tubing, a person may better see where they are going, may avoid tripping, and may avoid tripping over their oxygen tubing. This is especially important during nighttime hours or in dark corridors.

The lights that are used are also motion sensored, so that any movement of the patient or oxygen tubing will activate these lights. In addition, the lights are on a timer so that any touch or sense of movement will set off the lights for a predetermined period of time that is set by the user. This automatic illumination of the lights avoids any requirement to use cords, which may also be a tripping hazard. This automatic illumination also avoids the requirement that the user activates a switch every time that they move.

Once the device has remained stationary for a predetermined amount of time, it will automatically turn off. This allows the user to fall back asleep without having to remember to deactivate the lighting. In addition, this feature minimizes battery usage, and allows the lights to turn off without disturbing the user or a partner.

Oxygen tubing in the home if often fairly long, which allows it to stretch between rooms. This creates a tripping hazard in multiple rooms to everyone in the home, who is not on the constant lookout for oxygen tubing. Although one solution may be to only use portable oxygen containers, these have their own disadvantages due to their limited supply of oxygen. In addition, the portable oxygen containers themselves may have a long length of oxygen tubing, which may also create a trip hazard. Thus, illuminating the tubing so that it is bright and stands out is a practical solution to decrease the chances that a user or another person in the home may trip over the oxygen tubing.

In hospitals, patients may inadvertently lay on their oxygen tubing, which is often attached to a wall source. When this occurs, the oxygen tubing may kink, which restricts the flow of oxygen to the patient. It may not be readily apparent to either the user or a caretaker that the oxygen tubing is kinked, and not supplying the person the necessary amount of oxygen. By illuminating oxygen tubing, it is easier to see where it is, so that the patient wearing it can ensure that they are receiving the necessary supply of oxygen. The apparatus described in this patent application fulfills at least one of these needs or creates other utility.

### BRIEF DESCRIPTION OF THE DRAWINGS

The accompanying drawings, which are incorporated in and constitute a part of this specification, illustrate several aspects described below.

FIG. 1 is a left side view of the walker with an attached housing that may comprise one or more of the following: a microcontroller, a motion sensing device, a photo sensor, a timer, a power switch, a panic button, a temperature sensor, a proximity sensor, a GPS transmitter, and a battery, which is part of at least one of the embodiments of this invention.

FIG. 2 is a front view of the walker with an attached housing, which is part of at least one of the embodiments of this invention.

FIG. 3 is a top view of the walker with an attached housing, which is part of at least one of the embodiments of this invention.

FIG. 4 is a perspective view of the walker with an attached housing, which is part of at least one of the embodiments of this invention.

FIG. 5 is a front view of the walker with an attached housing, which is part of at least one of the embodiments of this invention.

FIG. 6 is a front view of part of the walker with an attached housing in which at least one of the embodiments of this invention is implemented.

FIG. 7 is a front view of part of the walker with an attached housing with lights attached to the outside in which at least one of the embodiments of this invention is implemented.

FIG. 8 is a left side view of the walker with an attached oxygen tubing and oxygen tank in which at least one of the embodiments of this invention is implemented.

FIG. 9 is a front view of the walker with an attached oxygen tubing and oxygen tank in which at least one of the embodiments of this invention is implemented.

FIG. 10 is a top view of the walker with an attached oxygen tubing and oxygen tank in which at least one of the embodiments of this invention is implemented.

FIG. 11 is a left perspective view of the walker with an attached oxygen tubing and oxygen tank in which at least one of the embodiments of this invention is implemented.

FIG. 12 is right perspective view of the walker with an attached oxygen tubing and oxygen tank in which at least one of the embodiments of this invention is implemented.

FIG. 13 is a close-up of the right perspective view of the walker with an attached oxygen tubing showing lights connected to the oxygen tubing, which is connected to an oxygen tank in which at least one of the embodiments of this invention is implemented.

### DETAILED DESCRIPTION OF THE INVENTION

It is to be understood that this invention is not limited to any particular embodiment described, which may vary. Also, it is to be understood that the terminology used herein is for the purpose of describing particular embodiments only, and is not intended to be limiting, since the scope of this invention will be limited only by the appended claims.

In the following detailed description, numerous specific details are set forth in order to explain and provide a thorough understanding of the present invention. However, it is apparent that the present invention may be practiced without all of these specific details. Thus, all illustrations of the drawings are for the purpose of describing versions of the present invention, and are not intended to limit the scope of the invention.

In the following section, the present invention is described fully by referencing the details in the enclosed drawings, which illustrate certain embodiments of the invention. The numbers shown in this specification refer to the corresponding numbers in the enclosed drawings. The terminology used is to describe the particular embodiment shown and is not intended to limit the scope of the invention. The invention may also be embodied in many other forms in addition to the embodiments shown. Thus, the embodiments shown should not be construed as limiting, but rather, to allow a thorough and complete description of the disclosure that conveys the scope of the invention to a person having ordinary skill in the art in the field of this invention. Therefore, for the terms used herein, the singular forms “the,” “a,” and “an” are intended to include the plural forms as well as the singular forms, unless the context clearly indicates otherwise. The term “and” includes any and all combinations of one or more of the associated listed items. As used herein, the terms “comprising” and “comprises” when used in this specification, identify specific steps, integers, operations, features, components, and elements, but do not preclude the presence or addition of one or more other steps, operations, features, components, and elements. In addition, the features, components, and elements referenced may be exaggerated for clarity.

Unless otherwise defined, all scientific terms, technical terms, or other terms used herein have the same meaning as the term that is understood by one having ordinary skill in the art in the field of this invention. It is also understood that these terms, including their dictionary meaning, should be understood as having the meaning, which is consistent with their definitions in the related relevant art. In addition, the present disclosure is not to be interpreted in an idealized or overly formal sense unless expressly stated so herein. Constructions or functions that are well known in the art may not be fully described in detail for brevity.

In describing the invention, it is understood that a number of steps and methods may be disclosed. Each of these may have individual benefit. Also, each may be used in conjunction with at least one or more of the disclosed steps and methods. Therefore, this description will refrain from stating each and every possible combination of the individual steps and methods for the sake of brevity. Regardless, the specification and related claims should be understood with the combinations that are entirely within the scope of the claims and inventions.

The disclosure in this invention are examples of how it may be implemented and are not intended to limit the scope of the invention to the specific embodiments shown in the accompanying drawings or the description provided herein. All illustrations are for the purpose of describing selected versions of the present invention and are not intended to limit the scope of the present invention. The present invention will now be described by example in the following paragraphs by referencing the accompanying drawings, which represent embodiments and alternative embodiments.

The present invention is an illuminated medical device comprising an illuminated oxygen tubing and illuminated attachments for walkers, wheelchairs, or the like. The illuminated oxygen tubing comprises a tubing that may be sized and shaped the same as standard oxygen tubing. The oxygen tubing may be composed of any transparent, flexible material which is medically safe.

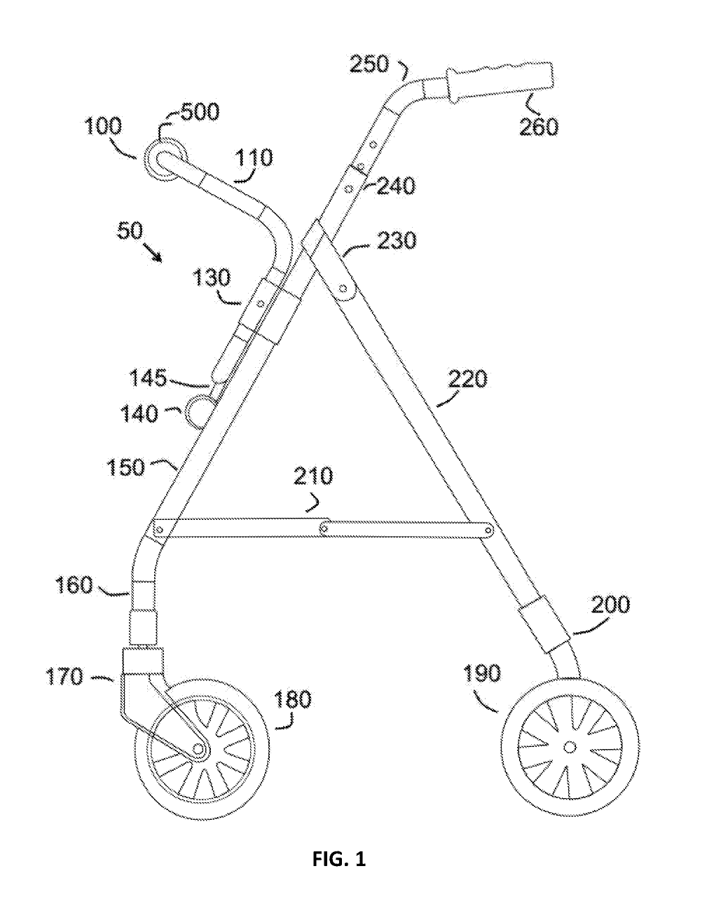

FIG. 1 shows a left side view of a walker 50. Left handgrip 260 connects to left handlebar 250. The height of left handlebar 250 can be adjusted through the connecting pin 240, which connects to the front frame of the walker frame 150. The front support 110 of the walker 50 connects to the front frame of the walker frame 150 with connecting clip 130. The front support 110 of the walker 50 has a foam pad 100 at its upper end. The front support 110 of the walker 50 allows the attachment of a housing 140 at its lower end. Wiring from the housing 140 travels through wire harness 145, which travels through front support 110 to a stub out 500 near foam pad 100 that allows for a power source for lights that may be attached to the oxygen tubing.

Also, as shown in FIG. 1, the bottom portion of the front bottom frame of the walker frame 160 connects to the front left fork 170, which connects to the front left wheel 180. The back of the frame 220 connects to the front frame of the walker 50 with connecting joint 230 and folding hinge 210. The bottom of the back frame of the walker frame 220 connects to the back left fork 200, which connects to the back left wheel 190.

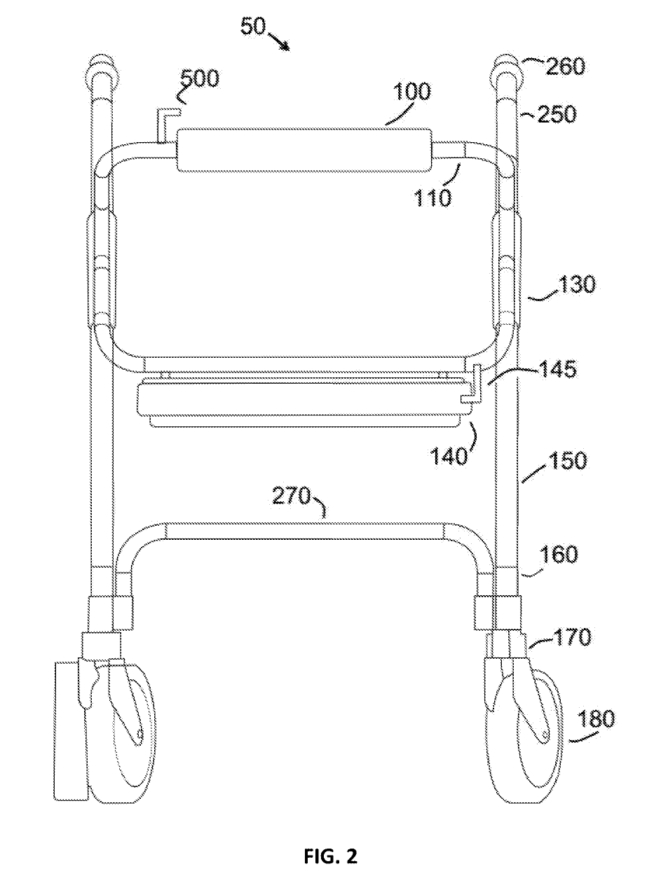

FIG. 2 is a front view of the walker 50. This figure also shows left handgrip 260 that connects to left handlebar 250. The front support 110 of the walker 50 uses clip 130 to connect to the front frame of the walker frame 150. The bottom of the front bottom frame of the walker frame 160 connects to the front left fork 170, which connects to the front left wheel 180. The bottom of the front bottom frame of the walker frame 160 also connects to the bottom horizontal support 270, which is also used to support the bottom of an oxygen tank 300, which is not shown in this figure.

The front support 110 allows the attachment of the housing 140 at its lower end that may comprise a microcontroller, a motion sensing device, a switch, a photo sensor, a timer, a battery, a panic button 800, a GPS transmitter, and a wireless communication device. The wiring from housing 140 travels through wiring harness 145 and the front support 110 to stub out 500, which is in the vicinity of the foam pad 100. Housing 140 is attached to walker 50 with a mounting system that may comprise a clamp, strap, hook, hanger, glue, Velcro, or similar device.

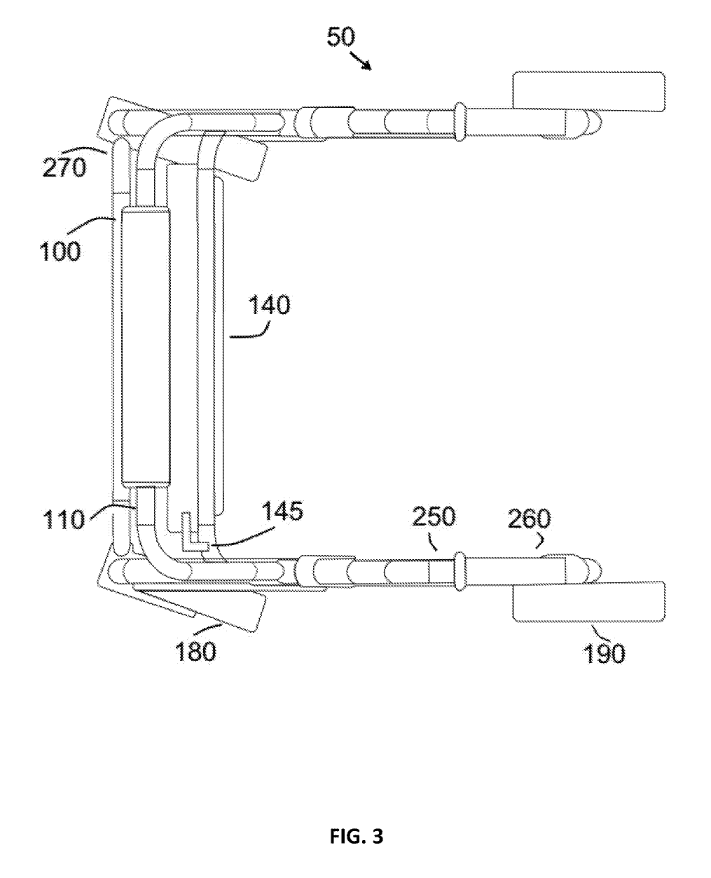

FIG. 3 is a top view of the walker 50. Left handgrip 260 connects to left handlebar 250. Also shown is the front left wheel 180, the back left wheel 190, the front support 110, foam pad 100, bottom horizontal support 270, housing 140, and wire harness 145.

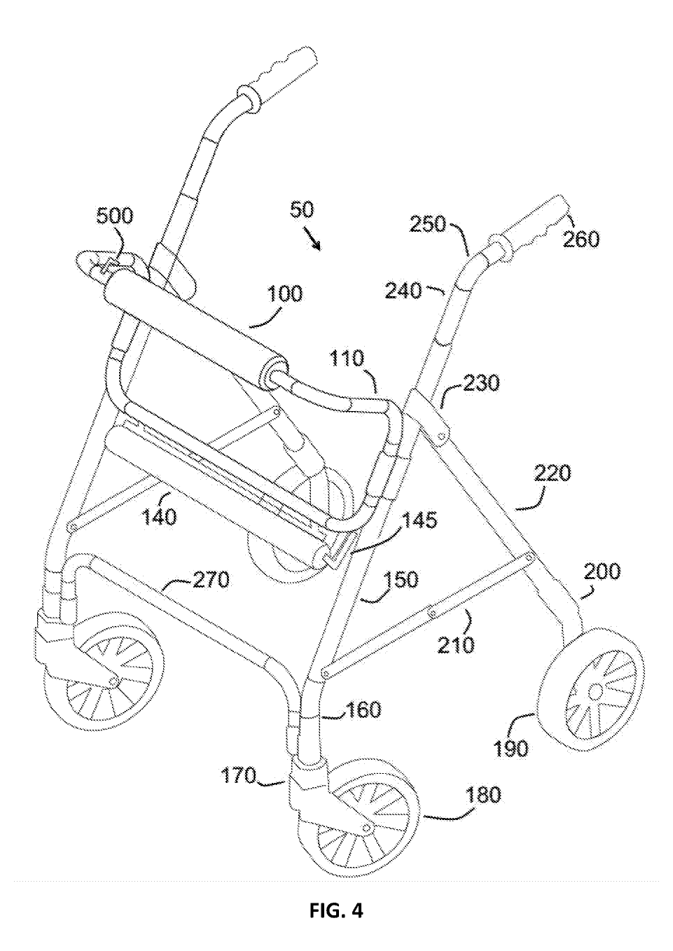

FIG. 4 is a perspective view of the walker 50. Left handgrip 260 connects to left handlebar 250, which can be adjusted through the connecting pin 240. The bottom horizontal support 270 connects to the bottom portion of the front bottom frame of the walker frame 160, which connects to the front left fork 170 that connects to the front left wheel 180. The back of the frame 220 connects to the front frame of the walker frame 150 with connecting joint 230 and folding hinge 210. The bottom of the back frame of the walker frame 220 also connects to the back left fork 200, which connects to the back left wheel 190. Also, shown is front support 110, foam pad 100, housing 140, wire harness 145, and wiring stub out 500.

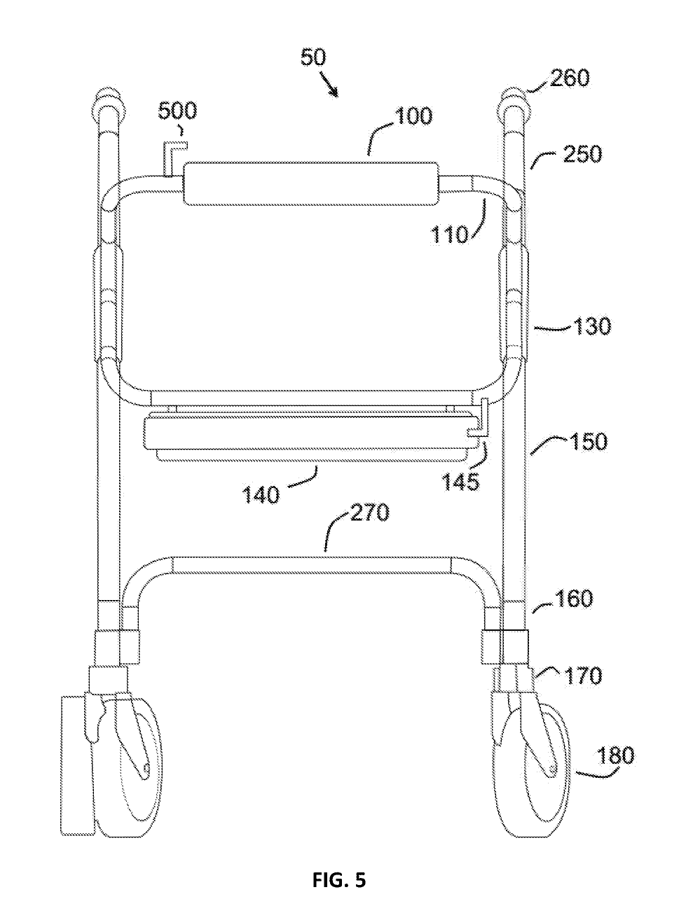

FIG. 5 is a front view of the walker 50. This figure also shows left handgrip 260 that connects to left handlebar 250. The front support 110 of the walker 50 connects to the front frame of the walker frame 150 with connecting clip 130. The front support 110 allows the attachment of housing 140 at its lower end and foam pad 100 at its upper end. Also shown is wire harness 145, which allows power to flow from the battery in the housing 140 to the wiring stub out 500, which may then connect to lights that may be attached to the oxygen tubing. The bottom horizontal support 270 connects to the bottom portion of the front bottom frame of the walker frame 160, which connects to the front left fork 170 that connects to the front left wheel 180.

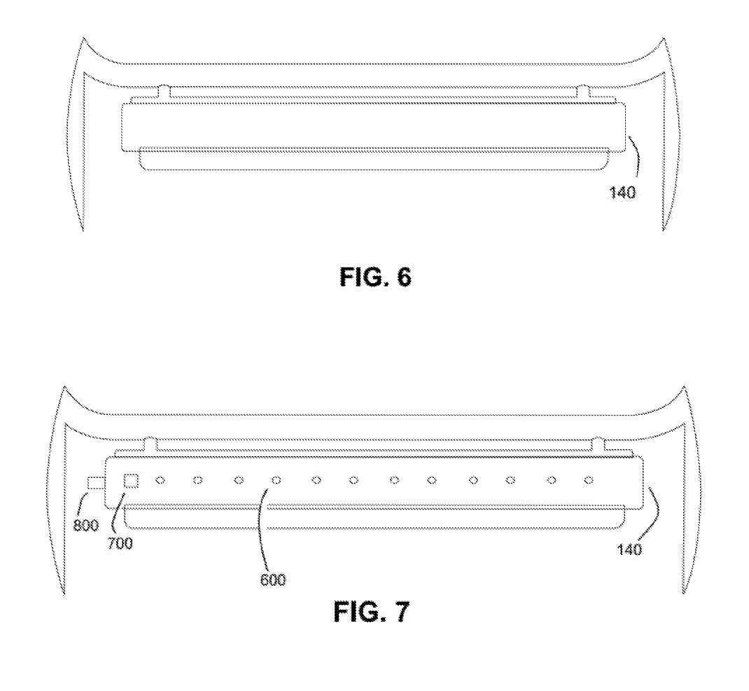

FIG. 6 is a front close-up view of the housing 140, which connects to the walker 50.

FIG. 7 is another front close-up view of the housing 140, which connects to the walker 50. In at least one embodiment, the plurality of lights 600 may be attached to the outside of the housing 140. The plurality of lights may be any type of lights. The lights may be selected from the group consisting of incandescent bulbs, LEDs, florescent, compact florescent, halogen, PAR lamps, MR lamps, and neon lamps. The housing 140 may also comprise a power switch 700 and a panic button 800.

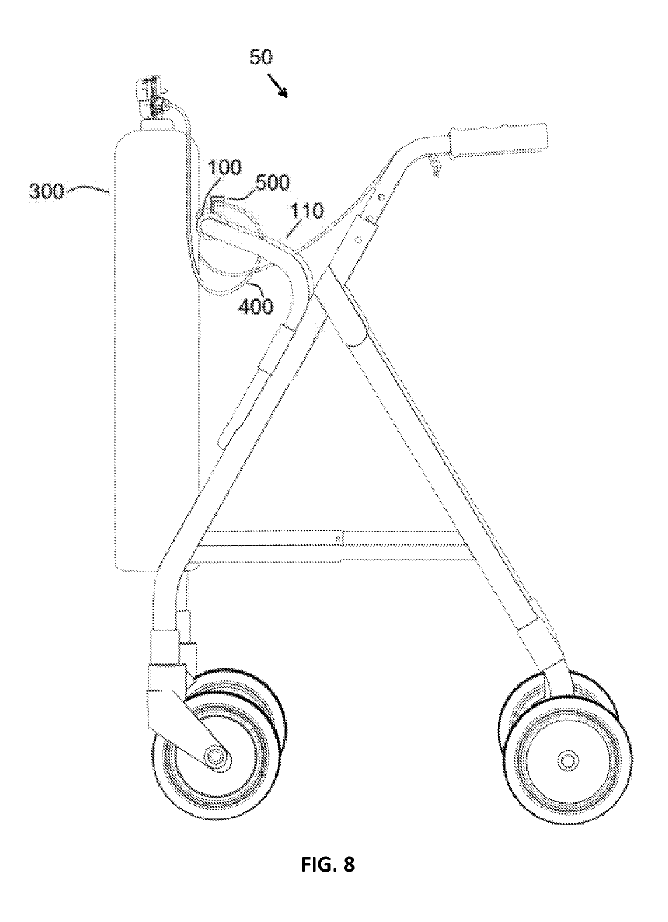

FIG. 8 shows a left side view of the walker 50 with an attached oxygen tank 300, oxygen tubing 400, and a wire harness stub out 500, to attach to a plurality of lights. The oxygen tubing 400 has a distal end and a proximal end. The distal end of the oxygen tubing is inserted into one or more of the user's nostrils, while the proximal end of the oxygen tubing connects to a source of oxygen. The oxygen tubing provides a means for delivering the oxygen to one or more of the user's nostrils. This figure also shows the front support 110 of the walker 50 and the foam pad 100 at its upper end.

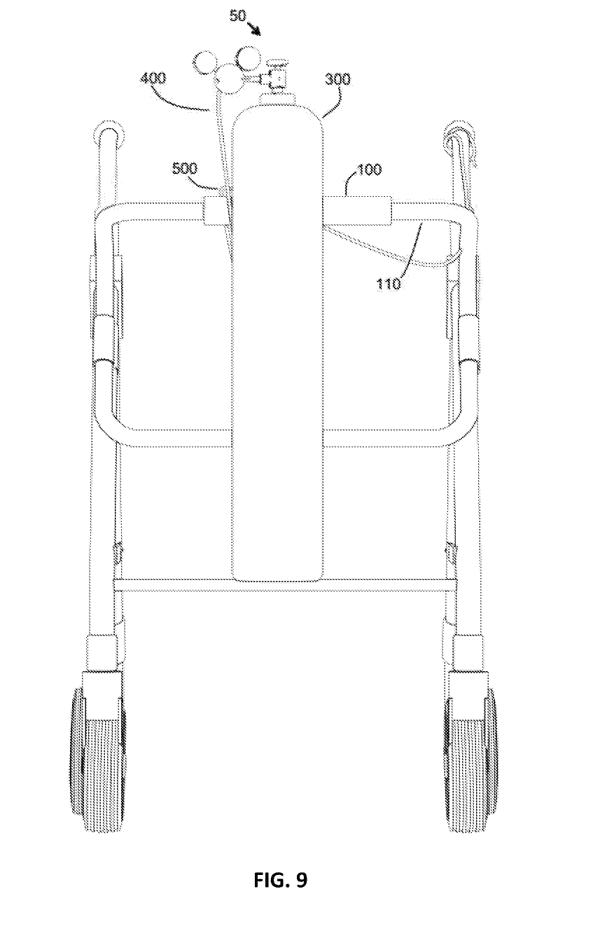

FIG. 9 is a front view of the walker 50 that also shows the location of the oxygen tank 300, the oxygen tubing 400, and the wire harness stub out 500 that attach to a plurality of lights. In addition, this figure also shows the front support 110 and the foam pad 100.

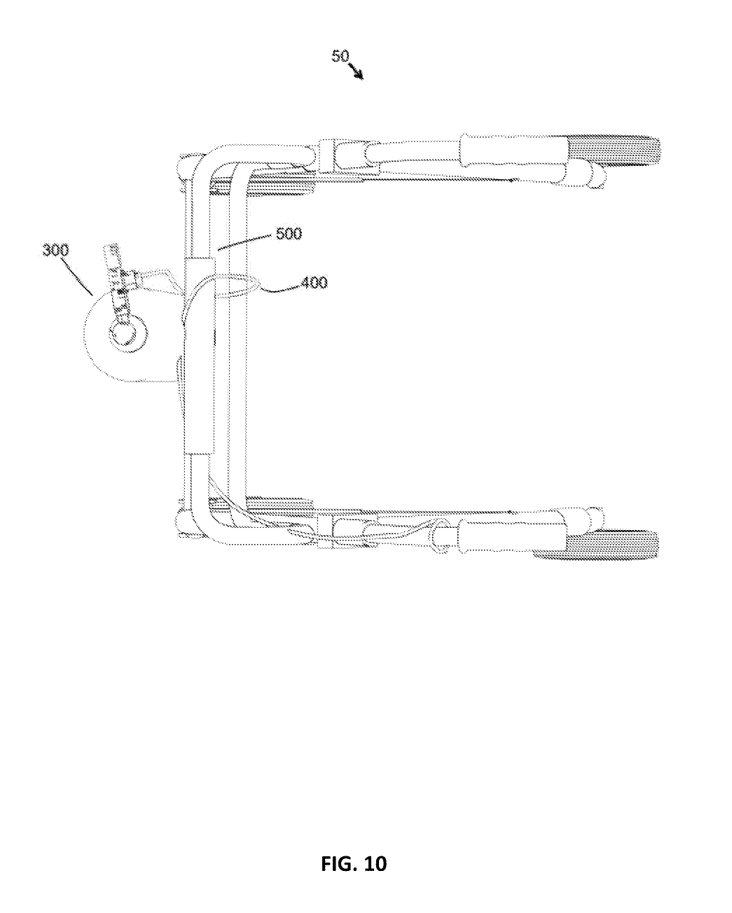

FIG. 10 is a top view of the walker 50 showing the location of the oxygen tank 300, the oxygen tubing 400, and the wire harness stub out 500.

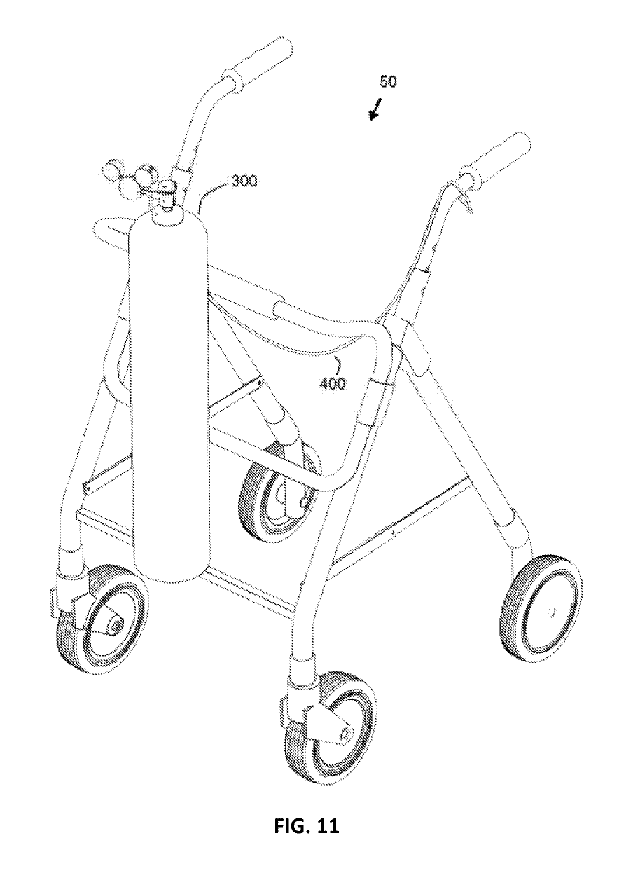

FIG. 11 is a left perspective view of the walker 50 showing the location of the oxygen tank 300 and the oxygen tubing 400.

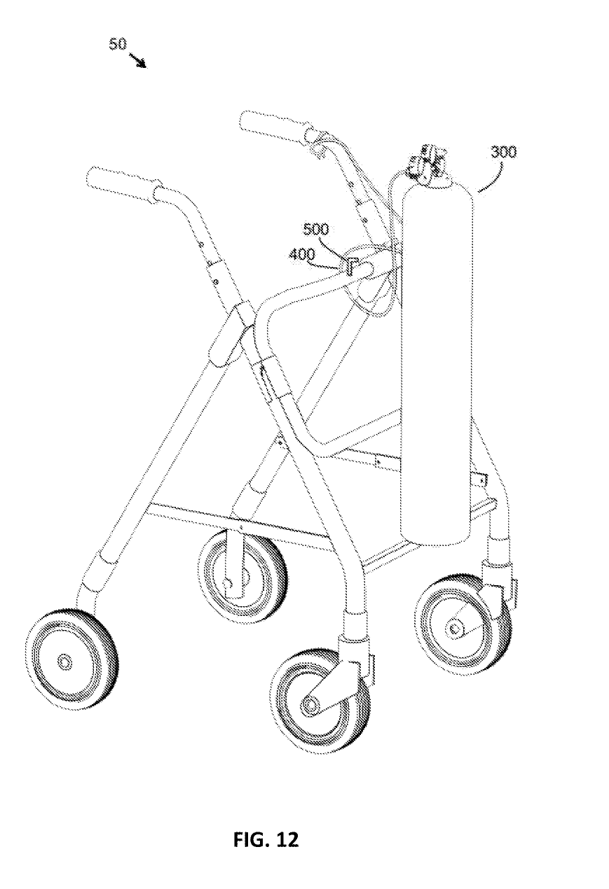

FIG. 12 is a right perspective view of the walker 50 showing the location of the oxygen tank 300, the oxygen tubing 400, and the wire harness stub out 500.

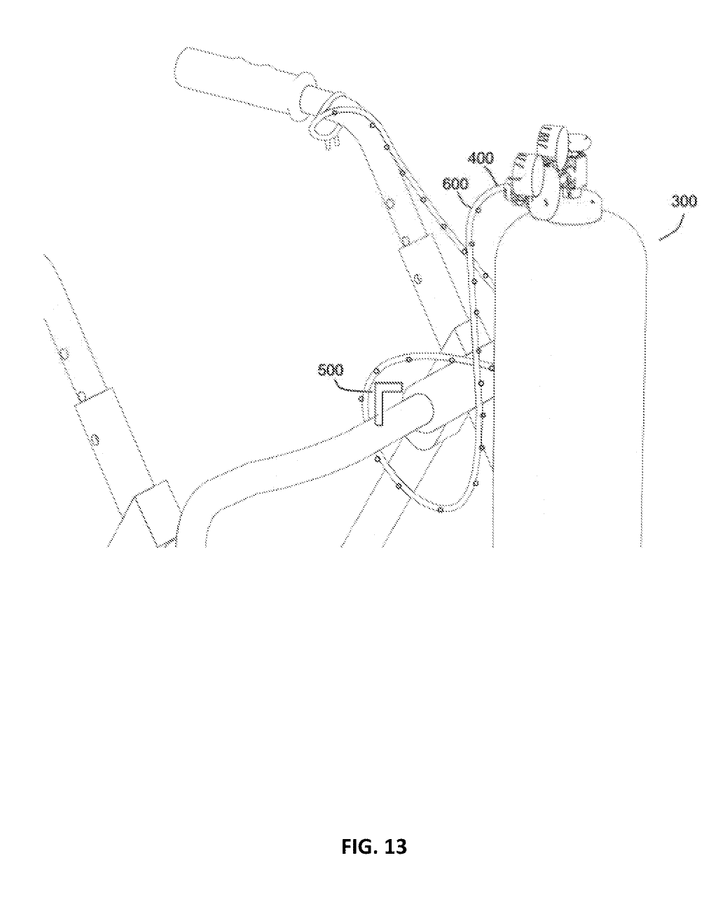

FIG. 13 is a close-up of the right perspective view of a light string comprising a plurality of lights 600. The lights 600 have a distal end and a proximal end, wherein the distal end of the lights connect to the distal end of the oxygen tubing 400, and the proximal end of the lights connect to the wiring harness stub out 500. The wiring harness stub out 500 connects to a battery in housing 140 through wires that run through the front frame 110, wherein the battery provides means for illuminating the lights 600.

Also, as shown in FIG. 12 and FIG. 13, in at least one embodiment of the invention, the illuminated lights 600 are attached to the oxygen tubing 400. The oxygen tubing 400 is connected to oxygen tank 300, which is connected to the walker 50. Electrical wiring connects the lights 600, which are attached to the lighted oxygen tubing 400, to the battery in the housing 140 through wire harness stub out 500. The plurality of lights are attached to the oxygen tubing 400 to illuminate areas that are dark or that have a low level of lighting. The lights 600 shown in FIG. 13 may be attached to the oxygen tubing 400 with a clamp, strap, hook, hanger, glue, Velcro, or similar device.

As shown in FIG. 1 through FIG. 7, the lights are powered and controlled by a battery in the housing 140, which may be of any shape or size and may be composed of a transparent material. The battery may be replaceable or rechargeable. In addition to the battery, the housing 140 may comprise wiring, a microcontroller, a motion sensing device, a photo sensor, a timer, a power switch 700, a panic button 800, a temperature sensor, a GPS transmitter, and a wireless communication device.

As shown in FIG. 7, in at least one embodiment, the housing 140 may contain a panic button 800, which may be located at the end of the housing 140. The patient could activate the panic button 800 if they feel light headed, dizzy, or need immediate assistance. The panic button 800 would use a GPS transmitter to send a signal through a wireless communication device to the hospital faculty. The hospital faculty would immediately determine the whereabouts of the patient through the GPS coordinates that would be transmitted by the GPS transmitter.

Also as shown in FIG. 7, in at least one embodiment, the housing 140 may contain a power switch 700. The power switch 700 may be positioned anywhere on the housing 140. In FIG. 7, the power switch 700 is shown at the end of a row of lights that may be optionally attached to the outside of the housing 140. The power switch 700 allows the user the manually turn on or off the plurality of lights. In at least one embodiment, as shown in FIG. 7, the lights may be positioned on housing 140.

In at least one embodiment, the power switch 700 or the panic button 800 or both may be located on the left handgrip 260, so that a user may have easy access to them, if this option is chosen, wiring would run from the housing 140 through the front frame of the walker frame 150, the connecting pin 240, the left handlebar 250, and into the left handgrip 260.

In at least one embodiment of the invention, the light string comprising the plurality of lights 600 is mounted within the oxygen tubing 400 to protect the lights from damage and to allow for a smooth outer surface of the oxygen tubing 400 that is less likely to snag on an obstacle.

In at least one embodiment of the invention wherein the light string comprising the plurality of lights 600 is encased in an outer tubing that slips over the oxygen tubing 400 to allow the lights 600 to be outside the oxygen tubing 400 while allowing for a smooth outer surface of the outer tubing that is less likely to snag on an obstacle.

In at least one embodiment of the invention, the lights 600 may be connected in a spiraling pattern around the circumference of the oxygen tubing 400 so that lights 600 are visible at equidistant intervals when viewing the oxygen tubing 400 from any position or angle.

In at least one embodiment of the invention, a spiraling pattern of lights 600 may be mounted approximately every 90 degrees around the circumference of the oxygen tubing 400. In this configuration, a light 600 is connected to the oxygen tubing 400 approximately every inch, so that there is a light 600 viewable at equidistant intervals of approximately every 4 inches when the oxygen tubing 400 is viewed from either its top, bottom, or either side.

In at least one embodiment of the present invention, the oxygen tubing 400 comprises an inner tubing that supplies oxygen, and a larger diameter surrounding tubing that provides a holding compartment for containing lights, such as LEDs. Thus, the holding compartment is the space between the outer wall of the inner tubing that contains oxygen and the inner wall of a surrounding outer tubing. The surrounding outer tubing may be transparent to allow the lights 600 to be visible that are between the inner wall of the surrounding tubing and the outer wall of the oxygen tubing 400. The holding compartment, which is created by the space between these two tubes, runs along the entire length of the oxygen tubing 400. The lights 600 within this holding compartment are connected together and powered by a battery, which may be mounted in the housing 140.

In at least one embodiment, the lights 600 may be connected to one or more sides of the housing 140 so that the lights 600 are visible when viewing the walker 50 from any position or angle. In at least one other embodiment, the lights 600 may be connected to other parts of the walker to illuminate the entire walker 50.

In at least one embodiment of the invention comprises at least one motion sensor. The motion sensor is wired to a power switch 700 in such a way that the power switch 700 is turned on when the motion sensor senses movement of the walker 50. When this occurs, a battery is turned on to power the lights 600 and illuminate the oxygen tubing 400. The lights then remain on for a predetermined amount of time as determined by a timer. The motion sensing device may be any motion sensor that is capable of detecting motion, such as an accelerometer.

At least one embodiment of the invention comprises at least one microcontroller. The microcontroller may be programmed to activate the plurality of lights 600 when the motion sensing device captures any motion or movement of the walker 50 or a device that is attached to the walker, such as the housing 140. The motion sensing device may be wired to the power switch 700 in such a way that when either the power switch 700 is turned on, or when the motion sensing device senses movement of the walker 50, the battery is powered on to illuminate the lights 600, which may be attached to the oxygen tubing 400.

At least one embodiment of the invention comprises at least one photo sensor. The photo sensor may be programmed to activate the lights 600 when it detects that the ambient brightness reaches a certain level of dimness, which may be measured in foot candles. The photo sensor may also be programmed to activate the lights 600 only when the motion sensor is measuring any motion or movement of the walker 50 or the device that it is attached to. In addition, the photo sensor may be programmed to keep the lights 600 on for a predetermined amount of time, which may be set by a timer, so that the lights do not immediately go off when there is a brief stoppage in the movement of the walker 50. After the brightness of the surrounding area rises above a predetermined level of dimness, the photo sensor turns the power switch 700 to the off position, which cuts power to the battery and the connected lights 600 causing the oxygen tubing 400 to no longer be illuminated.

At least one embodiment of the invention comprises at least one timer. The timer is activated when the power switch 700 is turned to an on position for a predetermined amount of time, which then allows the battery to power the lights 600 and illuminate the oxygen tubing 400. After the predetermined amount of time has elapsed, the timer turns the power switch 700 to the off position, which cuts power to the battery and the connected lights 600 causing the oxygen tubing 400 to no longer be illuminated.

In at least one embodiment, the housing 140 may contain an optional GPS transmitter. The GPS transmitter would assist the hospital faculty in determining the whereabouts of a patient. This could be especially helpful, if a patient travels outside the normal zone that they are expected to be in, such as an employee-only area of the hospital or outside the premises of the hospital. The GPS transmitter may also be configured to transmit an alarm through a wireless communication device, if the patient spends too much time in one area. For example, if the patient is detected to be on an elevator for longer than expected, the hospital faculty could be alerted that the patient may be trapped in an elevator. By the way of another example, if the patient is detected to be in a bathroom or another area for longer than expected, the hospital faculty could be alerted that the patient may have fallen in this area, are locked in the area, or are having difficulty in exiting this area.

At least one embodiment of the invention comprises at least one proximity sensor and means for mounting the proximity sensor to the oxygen tubing 400, wherein the proximity sensor is wired to the oxygen tubing 400 in such a way that the oxygen tubing 400 is turned on for a predetermined amount of time when the proximity sensor senses that it is getting close to an obstacle which causes the battery to power the lights 600 and illuminate the oxygen tubing 400.

At least one embodiment of the invention wherein the proximity sensor is coupled to a wireless communication device in the housing 140 and means for the wireless communication device to communicate a distress call to another wireless device if the proximity sensor contacts the floor or ground as may occur if the user falls.

At least one embodiment of the invention comprises a battery that is optionally coupled to a solar panel to assist in powering the battery. The battery may comprises a plug so that it can be plugged into an electric outlet to recharge it or to power any of the components in the housing 140.

At least one embodiment of the invention comprises at least one temperature sensor and means for mounting the temperature sensor to the oxygen tubing 400, wherein the temperature sensor is wired to the power switch 700 in such a way that the power switch 700 is turned on when the temperature sensor senses a temperature higher than a predetermined temperature or the usual room temperature which may occur if the patient lies on the oxygen tubing 400 and temperature sensor, or if a fire or failure of the room's HVAC system increases the surrounding air temperature to a dangerous amount.

At least one embodiment of the invention comprises a temperature sensor that is coupled to a wireless communication device in the vicinity of the user and means for the wireless communication device to communicate a distress call to another wireless device if the temperature sensor senses that the surrounding air temperature has reached an unsafe level.

All of these embodiments and the invention disclosed herein are intended to be within the scope herein disclosed. These and other embodiments of the invention will become readily apparent to those skilled in the art from the detailed description of the embodiments having reference to the attached figures, the embodiments not being limited to any particular embodiments disclosed. Also, the invention disclosed herein suitably may be practiced in the absence of any element which is not specifically disclosed herein.

Although the invention has been explained in relation to its at least one embodiment, embodiment, it is to be understood that many other possible modifications and variations can be made without departing from the spirit and scope of the invention as hereinafter claimed.

What is claimed is:

1. A device for illuminating oxygen tubing, the device comprising:
a. at least one piece of oxygen tubing, the oxygen tubing having a distal end and a proximal end, wherein the distal end of the oxygen tubing is inserted into one or more of the user's nostrils, and the proximal end of the oxygen tubing connects to a source of oxygen, wherein the oxygen tubing provides means for delivering the oxygen to one or more of the user's nostrils;
b. at least one light string comprising a plurality of lights, the light string having a distal end and a proximal end, wherein the distal end of the light string connects to the distal end of the oxygen tubing, and the proximal end of the light string connects to a battery, wherein the battery provides means for illuminating the light string;
c. at least one battery, the battery comprises a power switch and means for connecting the power switch, wherein the power switch provides means for turning the battery on and off;
d. at least one timer, the timer is activated when the power switch is turned to an on position for a predetermined amount of time, which then allows the battery to power the light string and illuminate the oxygen tubing, and after that amount of time has elapsed, the timer turns the power switch to the off position, which cuts power to the battery and the connected light string causing the oxygen tubing to no longer be illuminated;
e. at least one photo sensor, the photo sensor is activated when the brightness of the surrounding area falls below a predetermined level of dimness, which then allows the battery to power the light string and illuminate the oxygen tubing, and after the brightness of the surrounding area rises above a predetermined level of dimness, the photo sensor turns the power switch to the off position, which cuts power to the battery and the connected light string causing the oxygen tubing to no longer be illuminated;
f. at least one motion sensor and means for mounting the motion sensor to the oxygen tubing, wherein the motion sensor is wired to the power switch in such a way that the power switch is turned on for a predetermined amount of time when the motion sensor senses movement which causes the battery to power the light string and illuminate the oxygen tubing;
g. at least one motion sensing device that may be any motion sensor that is capable of detecting motion, such as an accelerometer, and means for mounting the motion sensing device to a walker, wherein the motion sensing device is wired to the power switch in such a way that the power switch is turned on when the motion sensing device senses movement of the walker which causes the battery to power the light string and illuminate the oxygen tubing;
h. at least one temperature sensor and means for mounting the temperature sensor to the oxygen tubing, wherein the temperature sensor is wired to the power switch in such a way that the power switch is turned on when the temperature sensor senses a temperature higher than a predetermined temperature or the usual room temperature which may occur if the patient lies on the oxygen tubing and temperature sensor, or if a fire or failure of the room's HVAC system increases the surrounding air temperature to a dangerous amount;
i. at least one proximity sensor and means for mounting the proximity sensor to the oxygen tubing, wherein the proximity sensor is wired to the power switch in such a way that the power switch is turned on for a predetermined amount of time when the proximity sensor senses that it is getting close to an obstacle which causes the battery to power the light string and illuminate the oxygen tubing;
j. at least one GPS transmitter and means for sending a signal to a remote location for immediate assistance, wherein the GPS transmitter provides communication over a wireless medium from a group consisting of wifi, cellular, Bluetooth, or satellite communications;
k. at least one panic button, the panic button comprises a button and means for connecting the button to a GPS transmitter to send a signal for immediate assistance, wherein the panic button provides means for activating the GPS transmitter.

2. The device of claim 1, wherein the battery is optionally coupled to a solar panel to assist in powering the battery.

3. The device of claim 1, wherein the battery comprises a plug so that it can be plugged into an electric outlet while still powering the light string.

4. The device of claim 1, wherein the lights on the light string are selected from the group consisting of incandescent bulbs, LEDs, florescent, compact florescent, halogen, PAR lamps, MR lamps, and neon lamps.

5. The device of claim 1, wherein the lights on the light string are mounted within the oxygen tubing to protect the lights from damage and to allow for a smooth outer surface of the oxygen tubing that is less likely to snag on an obstacle.

6. The device of claim 1, wherein the lights on the light string are encased in an outer tubing that slips over the oxygen tubing to allow the lights to be outside the oxygen tubing while allowing for a smooth outer surface of the outer tubing that is less likely to snag on an obstacle.

7. The device of claim 1, wherein the proximity sensor is coupled to a wireless communication device in the vicinity of the head of the user and means for the wireless communication device to communicate a distress call to another wireless device if the proximity sensor contacts the floor or ground as may occur if the user falls.

8. The device of claim 1, wherein the temperature sensor is coupled to a wireless communication device in the vicinity of the user and means for the wireless communication device to communicate a distress call to another wireless device if the temperature sensor senses that the surrounding air temperature has reached an unsafe level.

9. The device of claim 1, wherein the lights are connected in a spiraling pattern around the circumference of the oxygen tubing so that the lights on the light string are visible at equidistant intervals when viewing the oxygen tubing from any position or angle.

10. The device of claim 1, wherein the lights are connected in a spiraling pattern that locates a light on the light string approximately every 90 degrees around the circumference of the oxygen tubing and a light on the light string is connected to the oxygen tubing approximately every inch so that there is a light viewable at an equidistant intervals of approximately every 4 inches when the oxygen tubing is viewed from its top, bottom, or either side.

11. A device for illuminating a walker, the device comprising:
a. at least one housing, wherein the housing is attached to a walker;
b. at least one light, wherein the lights are attached to the housing;
c. at least one battery, wherein the battery is encased in the housing, wherein the battery provides power for illuminating the lights and for operating other components;
d. at least one power switch, wherein the power switch provides means for turning the battery on and off;
e. at least one timer, wherein the timer is activated when the power switch is turned to an on position for a predetermined amount of time, which then allows the battery to power the lights, and after that amount of time has elapsed, the timer turns the power switch to the off position, which cuts power to the battery and the connected lights causing the lights to no longer be illuminated;
f. at least one photo sensor, wherein the photo sensor is activated when the brightness of the surrounding area falls below a predetermined level of dimness, which then allows the battery to power the lights and illuminate the area in front of the walker, and after the brightness of the surrounding area rises above a predetermined level of dimness, the photo sensor turns the power switch to the off position, which cuts power to the battery and the connected lights causing the lights to no longer be illuminated;
g. at least one motion sensor and means for mounting the motion sensor to the lights, wherein the motion sensor is wired to the power switch in such a way that the power switch is turned on for a predetermined amount of time when the motion sensor senses movement which causes the battery to power the lights and illuminate the area in front of the walker;
h. at least one motion sensing device that may be any motion sensor that is capable of detecting motion, such as an accelerometer, and means for mounting the motion sensing device to a walker, wherein the motion sensing device is wired to the power switch in such a way that the power switch is turned on when the motion sensing device senses movement of the walker which causes the battery to power the lights and illuminate the area in front of the walker;
i. at least one temperature sensor and means for mounting the temperature sensor to the housing, wherein the temperature sensor is wired to the power switch in such a way that the power switch is turned on when the temperature sensor senses a temperature higher than a predetermined temperature or the usual room temperature which may occur if a fire or failure of the room's HVAC system increases the surrounding air temperature to a dangerous amount;
j. at least one proximity sensor and means for mounting the proximity sensor to the housing, wherein the proximity sensor is wired to the power switch in such a way that the power switch is turned on for a predetermined amount of time when the proximity sensor senses that it is getting close to an obstacle which causes the battery to power the lights and illuminate the area in front of the walker;
k. at least one GPS transmitter and means for sending a signal to a remote location for immediate assistance, wherein the GPS transmitter provides communication over a wireless medium from a group consisting of wife, cellular, Bluetooth, or satellite communications;
l. at least one panic button, the panic button comprises a button and means for connecting the button to a GPS transmitter to send a signal for immediate assistance, wherein the panic button provides means for activating the GPS transmitter.

12. The device of claim 11, wherein the battery is optionally coupled to a solar panel to assist in powering the battery.

13. The device of claim 11, wherein the battery comprises a plug so that it can be plugged into an electric outlet while still powering the lights.

14. The device of claim 11, wherein the lights are selected from the group consisting of incandescent bulbs, LEDs, florescent, compact florescent, halogen, PAR lamps, MR lamps, and neon lamps.

15. The device of claim 11, wherein the lights are mounted within the oxygen tubing to protect the lights from damage and to allow for a smooth outer surface of the oxygen tubing that is less likely to snag on an obstacle.

16. The device of claim 11, wherein the lights are encased inside of the housing, wherein the housing is made from a transparent material.

17. The device of claim 11, wherein the proximity sensor is coupled to a wireless communication device in the housing and means for the wireless communication device to communicate a distress call to another wireless device if the proximity sensor contacts the floor or ground as may occur if the user falls.

18. The device of claim 11, wherein the temperature sensor is coupled to a wireless communication device in the housing and means for the wireless communication device to communicate a distress call to another wireless device if the temperature sensor senses that the surrounding air temperature has reached an unsafe level.

19. The device of claim 11, wherein the lights are connected to one or more sides of the housing so that the lights are visible when viewing the walker from any position or angle.

20. The device of claim 11, wherein the lights are connected to other parts of the walker to illuminate the entire walker.
